(19) United States
(12) Patent Application Publication
(10) Pub. No.: US 2024/0017042 A1
(43) Pub. Date: Jan. 18, 2024

(54) Blood Collection Device with Variable-Diameter Fluid Flow Conduit

(71) Applicant: Becton, Dickinson and Company, Franklin Lakes, NJ (US)

(72) Inventors: Alison Nicole Thompson, Salt Lake City, UT (US); Curtis H. Blanchard, Herriman, UT (US); Jonathan Karl Burkholz, Salt Lake City, UT (US); Yiping Ma, Layton, UT (US); Weston F. Harding, Lehi, UT (US); Justin G. Hortin, Farmington, UT (US)

(21) Appl. No.: 18/221,655

(22) Filed: Jul. 13, 2023

Related U.S. Application Data
(60) Provisional application No. 63/389,183, filed on Jul. 14, 2022.

(51) Int. Cl. A61M 25/06; A61M 25/00

(52) U.S. Cl. CPC A61M 25/0606; A61M 25/0097

(57) ABSTRACT

A blood draw device for withdrawing blood through an intravenous catheter assembly including a catheter, the blood draw device including a housing, a fluid conduit received within the housing and having a proximal end and a distal end, the fluid conduit including a first conduit portion at the proximal end of the fluid conduit, the first conduit portion having a proximal end, a distal end, and a first diameter, and a second conduit portion at the distal end of the fluid conduit, the second conduit portion having a proximal end, a distal end, and a second diameter different from the first diameter, wherein the fluid conduit is configured to be advanced from a proximal position in which the second conduit portion does not extend beyond a distal end of the catheter to a distal position in which the second conduit portion extends beyond a distal end of the catheter.

### CROSS-REFERENCE TO RELATED APPLICATION

The present application claims priority to U.S. Provisional Application No. 63/389,183 entitled “Blood Collection Device with Variable-Diameter Fluid Flow Conduit” filed Jul. 14, 2022, the entire disclosure of which is hereby incorporated by reference in its entirety.

### BACKGROUND OF THE INVENTION

### Field of the Invention

Provided herein are devices and systems for use in vascular access, and, in particular, devices and systems for use with blood draw through indwelling peripheral catheters.

### Description of Related Art

A vascular access device (VAD) may access peripheral vasculature of a patient. A VAD may be indwelling for short term (days), moderate term (weeks), or long term (months to years). VADs may be used for infusion therapy and/or for blood withdrawal.

A common type of VAD is an over-the-needle peripheral intravenous catheter (PIVC). Currently, there may be several limitations to the use of a PIVC for fluid infusion or blood draw. The PIVC or vein may narrow, collapse, or clog with time, leading to failure of the PIVC. Also, small lumen sizes required for devices used in withdrawing blood through a PIVC can result in low flow rates, and clogging, rendering the process somewhat inefficient. Accordingly, there is a need in the art for devices and systems that allow for higher flow rates of blood during blood withdrawal through PIVCs.

### SUMMARY OF THE INVENTION

Provided herein is a blood draw device for withdrawing blood through an intravenous catheter assembly including a catheter, the blood draw device including a housing, a fluid conduit received within the housing and having a proximal end and a distal end, the fluid conduit including a first conduit portion at the proximal end of the fluid conduit, the first conduit portion having a proximal end, a distal end, and a first diameter, and a second conduit portion at the distal end of the fluid conduit, the second conduit portion having a proximal end, a distal end, and a second diameter different from the first diameter, wherein the fluid conduit is configured to be advanced from a proximal position in which the second conduit portion does not extend beyond a distal end of the catheter to a distal position in which the second conduit portion extends beyond a distal end of the catheter.

Also provided herein is a catheter system including a catheter assembly including a catheter adapter, having a distal end, a proximal end, a lumen extending between the distal end and the proximal end and a catheter secured to the catheter adapter and extending distally from the catheter adapter, wherein the catheter has a distal end and a proximal end, and a blood draw device for withdrawing blood through the intravenous catheter assembly, the blood draw device including a housing, a fluid conduit received within the housing and having a proximal end and a distal end, the fluid conduit including a first conduit portion at the proximal end of the fluid conduit, the first conduit portion having a proximal end, a distal end, and a first diameter, and a second conduit portion at the distal end of the fluid conduit, the second conduit portion having a proximal end, a distal end, and a second diameter different from the first diameter, wherein the fluid conduit is configured to be advanced from a proximal position in which the second conduit portion does not extend beyond the distal end of the catheter to a distal position in which the second conduit portion extends beyond the distal end of the catheter.

### BRIEF DESCRIPTION OF THE DRAWINGS

FIG. 1 shows a system including a vascular access device and blood draw device according to non-limiting embodiments described herein;

FIGS. 2A-2D show a variable-diameter fluid for a blood draw device according to non-limiting embodiments described herein;

FIGS. 3A and 3B show a variable-diameter fluid for a blood draw device according to non-limiting embodiments described herein;

FIGS. 4A and 4B show a variable-diameter fluid for a blood draw device according to non-limiting embodiments described herein;

FIG. 5 shows a variable-diameter fluid for a blood draw device according to non-limiting embodiments described herein;

FIG. 6 shows a variable-diameter fluid for a blood draw device according to non-limiting embodiments described herein;

FIG. 7 shows a variable-diameter fluid for a blood draw device according to non-limiting embodiments described herein; and

FIG. 8 shows a variable-diameter fluid for a blood draw device according to non-limiting embodiments described herein.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

It should be understood that any numerical range recited herein is intended to include all values and sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

Provided herein are devices and systems for blood draw through indwelling catheters, such as peripheral intravenous catheters (PIVCs). While certain blood draw devices are shown in the accompanying figures and described below, those of skill will appreciate that the variable-diameter fluid conduits described herein may be useful in any number of different blood draw devices where a smaller diameter conduit is useful and/or necessary.

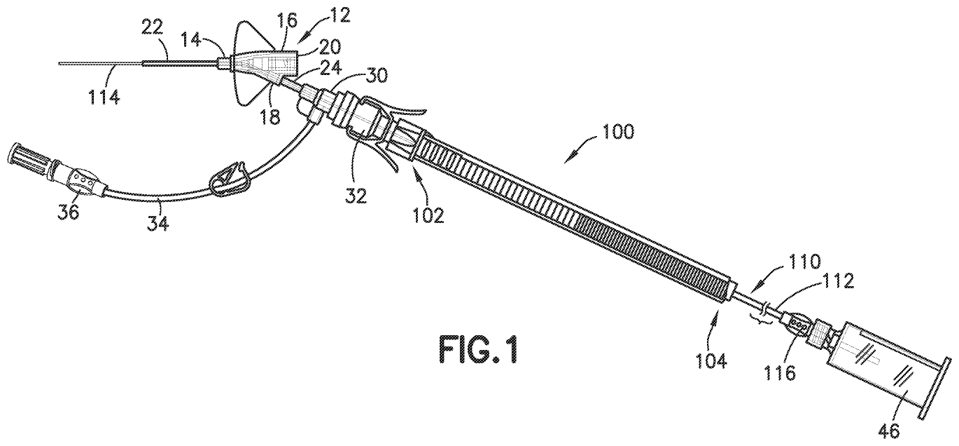

Turning to FIG. 1, shown is a system with a catheter assembly 10 and a blood draw device 100. The catheter assembly 10 may include a catheter adapter 12, which may include a distal end 14 and a proximal end 16. In some embodiments, the catheter adapter 12 may include an additional port 18. In some embodiments, port 18 may disposed between the distal end 14 and the proximal end 16. In some embodiments, port 18 may be disposed at proximal end 16. In some embodiments, the first catheter adapter 12 may include a first lumen 20 extending through the distal end 14 and the proximal end 16.

In some non-limiting embodiments or aspects, the catheter assembly 10 may include a catheter 22 extending from the distal end 14. In some embodiments, the first catheter 22 may include a peripheral intravenous catheter, a midline catheter, or a peripherally-inserted central catheter. Catheter 22 may be formed of any suitable material and may be of any useful length, as known to those of skill in the art. In some non-limiting embodiments or aspects, the catheter assembly 10 may include a first fluid conduit 24 extending from the port 18. First fluid conduit 24 may be formed of any suitable material known to those of skill in the art. In some non-limiting embodiments or aspects, a connector 30 may be coupled to first fluid conduit 24. Connector 30 may be a t-connector (e.g., one side port arranged at a 90 degree angle relative to a longitudinal axis of connector 30), a y-connector (e.g., one side port arranged at a 25, a 60, or a 75 degree angle relative to a longitudinal axis of connector 30), or any other type of connector known in the art, and may include a second lumen therethrough, having any number of branches suitable for the type of connector.

In some non-limiting embodiments or aspects, catheter assembly 10 may include an extension set including a second fluid conduit 34. In non-limiting embodiments, connector 30 includes a port to which an extension set (e.g., second fluid conduit 34) may be connected. Extension sets 34 are known to those of skill in the art and are commercially available from, for example, Becton, Dickinson and Company under the tradenames MAXPLUS, MAXZERO, NEUTRACLEAR, Q-SYTE, and SMARTSITE. In some non-limiting embodiments or aspects, second fluid conduit 34 may include a luer connection 36 at an end thereof. In some non-limiting embodiments or aspects, the extension set may include a clamp 40, to allow for occlusion of second fluid conduit 34. Clamp 40 and second fluid conduit 34 may be formed of any suitable materials known to those of skill in the art.

Catheter assembly 10 may include a needleless access connector 32. Needleless access connectors 32 are known to those of skill in the art and are commercially available from, for example, Becton, Dickinson and Company under the tradenames MAXPLUS, MAXZERO, Q-SYTE, and SMARTSITE.

With continuing reference to FIG. 1, also shown is a blood draw device 100, having a distal end 102 and a proximal end 104. Exemplary blood draw devices are described in, for example, U.S. Patent Application Publication No. 2020/0316346 and U.S. Pat. No. 10,300,247, the contents of which are incorporated herein by reference in their entirety. Blood draw device 100 may include, at distal end 102 thereof, one or more structures for reversibly coupling to catheter assembly 10, for example via needless access connector 32 as shown in FIG. 1. Blood draw device 100 includes a fluid flow conduit 110 received at least partially within a housing of the device 100. In non-limiting embodiments, conduit 110 is movably received within blood draw device 100, such that, when device 100 is coupled to a catheter assembly, conduit 110 is advanceable through catheter adapter 12 and catheter 22, to enter a patient's vasculature. As will be described in greater detail below, conduit 110 may include a first conduit portion 112 and a second conduit portion 114. First conduit portion 112 and second conduit portion 114 may be formed of any suitable materials, including polymeric materials, metals, alloys, and combinations thereof. Blood draw device 100 may include, at a proximal end of first conduit portion 112, a luer connector 116 for reversibly coupling to one or more devices to effectuate blood draw, such as a luer lock adapter (LLA), e.g., LLA 46 as shown in FIG. 1. LLA 46 may receive a syringe and/or an evacuated container for withdrawing blood through conduit 110.

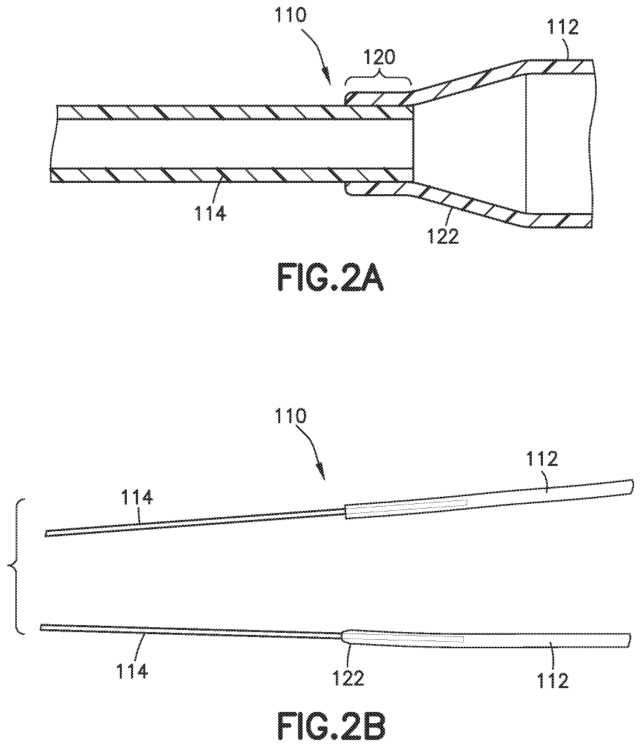

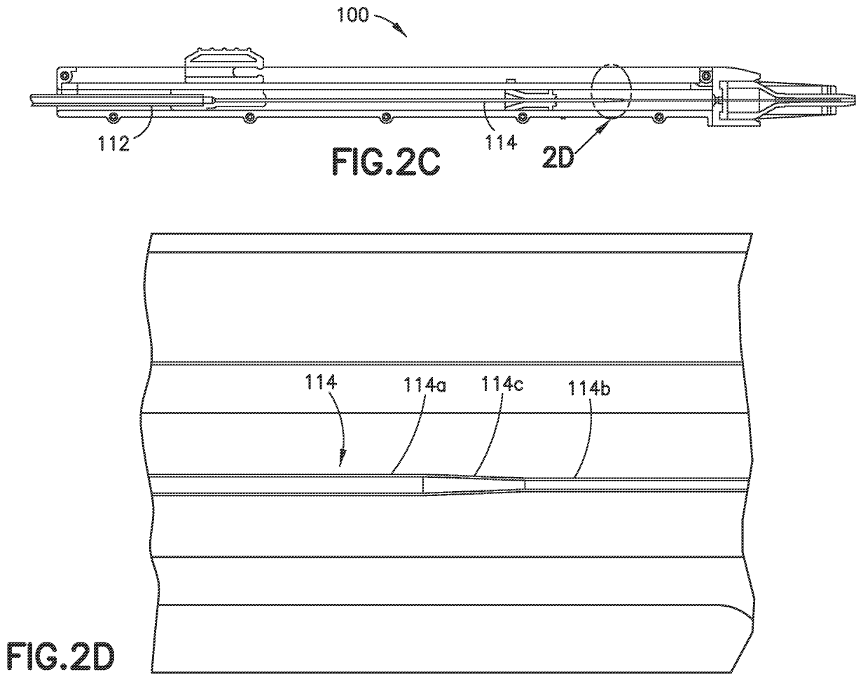

Turning to FIGS. 2A-8, as described above, conduit 110 may include a first conduit portion 112 and a second conduit portion 114. In non-limiting embodiments, first conduit portion 112 is of a larger diameter than second conduit portion 114, to increase flow rate and to simultaneously allow passage through variable inner geometries of an IV catheter, as set forth in Formula 1 below:

$$Q = \frac{π {Pr}^{4}}{8 η l} ,$$

where Q is flow rate, P is pressure, r is radius, η is fluid viscosity, and l is the length of the tubing. By increasing the radius of the tubing, for example by use of larger diameter first conduit portion 112, flow rate can be increased.

Those of skill will appreciate, in particular with reference to the accompanying figures, that while the term “conduit portion” is used herein, each conduit portion may be a distinct fluid conduit, each formed of the same, or distinct, materials. One or both of first conduit portion 112 and second conduit portion 114 may be formed of materials including polyurethanes, polyimides, or other suitable materials known to those of skill in the art.

With regard to FIGS. 2A and 2B, shown is a non-limiting embodiment in which conduit 110 includes first conduit portion 112 having at least a first diameter and second conduit portion 114 having a second diameter, where second diameter is less than the first diameter. First conduit portion 112 may overlap with second conduit portion 114 for a first distance 120. First fluid conduit 112 may include a taper 122 to a tip region (shown, in FIG. 2A, as being coextensive with area of overlap 120) having a smaller diameter, where, in non-limiting embodiments, second conduit portion 114 may be received in a fluid-impermeable manner. At one or more locations along area of overlap 120, first conduit portion 112 and second conduit portion 114 may be bonded together, for example by heat shrinking, through use of one or more adhesives, welding, and/or other known processes for joining two structures in a fluid-tight manner. FIG. 2B shows two different embodiments, one with the taper and tip region and one without.

With regard to FIGS. 2C and 2D, shown is a non-limiting embodiment of conduit 110 for use with a blood draw device 100, in which second conduit portion 114 itself includes a number of portions having different inner diameters. While an arrangement in which second conduit portion 114 includes a larger diameter portion 114a, smaller diameter portion 114b, and transition 114c, is exemplified, those of skill in the art will appreciate that in order to increase the diameter of conduit 110 as quickly as possible to increase flow rate (as described above), any number of steps in terms of conduit diameter, with or without transitions of varying rate of diameter increase, may be included at any suitable position along second conduit portion 114 and/or first conduit portion 112. Further, at any junction between portions of conduit having different diameters (e.g., between 114a and 114b), any transition or joining described herein may be used.

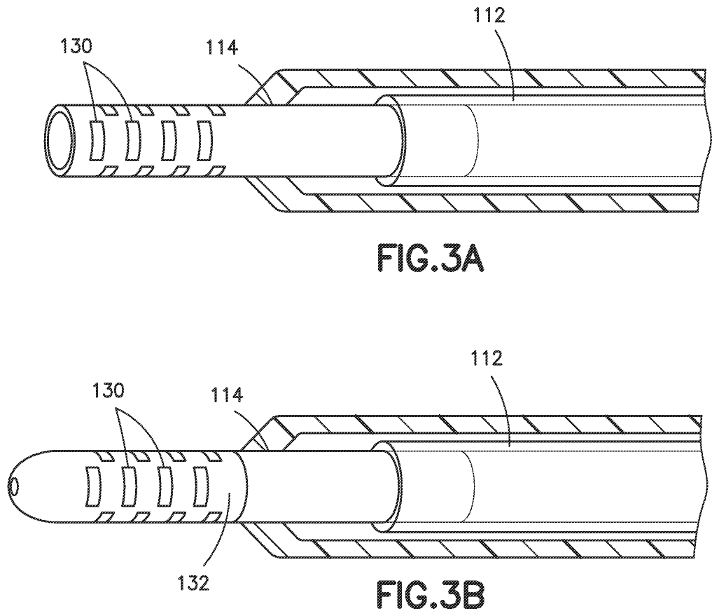

Turning to FIGS. 3A and 3B, in embodiments of the conduits disclosed herein, a distal end of second conduit portion 114 may include one or more openings 130, for example in a sidewall thereof. As shown in FIG. 3A, in non-limiting embodiments one or more openings 130 are formed directly in second conduit portion. In non-limiting embodiments, for example as shown in FIG. 3B, one or more openings 130 may be included in a third conduit portion 132, arranged at a distal end of second conduit portion 114. Third conduit portion 132 may be formed of nitinol or other materials described herein or known to those of skill in the art, and may be bound to second conduit portion 114 by known means, including those described herein. In non-limiting embodiments, second conduit portion 114 and/or third conduit portion 132 are formed of stainless steel or nitinol, and may be bound to second conduit portion 114 and/or first conduit portion 112 by known means. In non-limiting embodiments, third conduit portion 132 includes an atraumatic tip (e.g., rounded) to lessen the likelihood of damage to the vasculature.

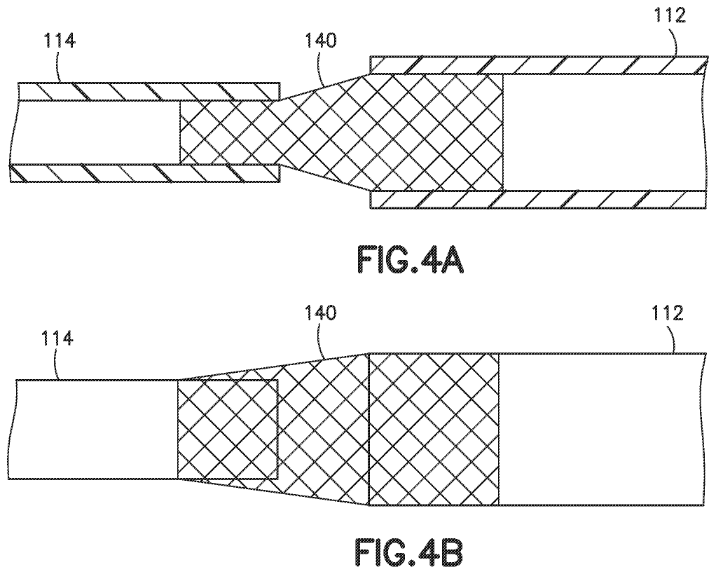

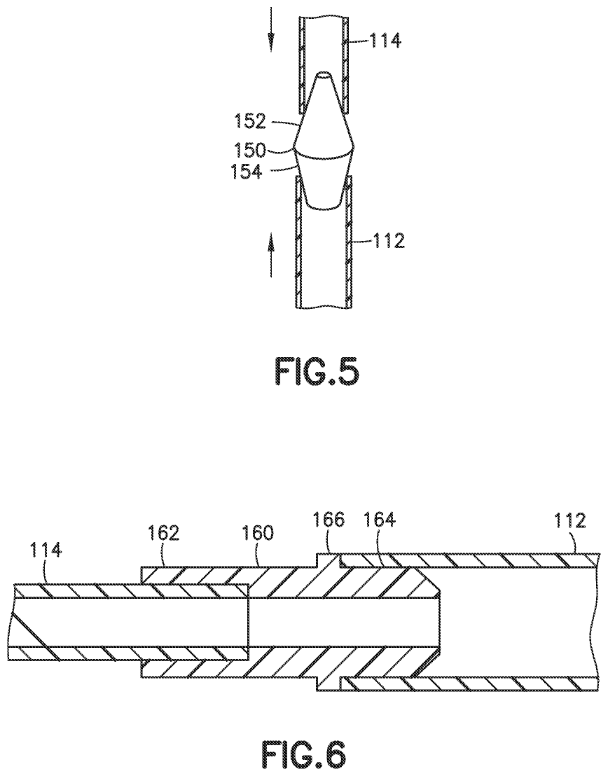

Turning to FIGS. 4A-6, in non-limiting embodiments, first conduit portion 112 and second conduit portion 114 may be bound to one another through use of a joining device. Such a joining device may take the form of, for example, a sleeve, such as an expandable sleeve 140 (FIG. 4), a wedge 150 (FIG. 5), and/or a bushing 160 (FIG. 6), and may provide a fluid-tight connection between first conduit portion 112 and second conduit portion 114. Each of the sleeve 140, wedge 150, and bushing 160 may be formed of any suitable material, for example, metals, alloys (e.g., nitinol), plastics, and the like, and each joining device may be coupled to first conduit portion 112 and/or second conduit portion 114 by known methods, for example those disclosed herein. While certain arrangements of the joining device(s) are shown in FIGS. 4A-6, those of skill will appreciate that any joining device may be arranged on the inside of, or about an outer surface of, first conduit portion 112 and/or second conduit portion 114.

With regard to FIGS. 4A-4B, as noted above sleeve 140 may be formed of any suitable material (in non-limiting embodiments, nitinol), and may be of any suitable length and any arrangement in terms of length and distance between struts, angle of helices, and like parameters known to those of skill in the art. Sleeve 140 may be arranged in the interior of first conduit portion 112 and/or second conduit portion 114 (FIG. 4A) and/or about the exterior of first conduit portion 112 and/or second conduit portion 114 (FIG. 4B). In non-limiting embodiments, sleeve 140 is joined to first conduit portion 112 and/or second conduit portion 114 through use of an adhesive.

With regard to FIG. 5, wedge 150 as noted above may be formed of any suitable material, and may include one or more flared portions 152, 154. Flared portions 152, 154 may be frustoconical in shape. Flared portion(s) 152, 154 may be of different angles, for joining first fluid conduit 112, having a larger diameter, to second fluid conduit 114, having a smaller diameter. For example flared portion 152 may be configured such that it may be received within the smaller inner diameter of second conduit portion 114, while flared portion 154 may be configured such that it may be received within the larger inner diameter of first conduit portion 112.

With regard to FIG. 6, bushing 160 as noted above may be formed of any suitable material, and may include a proximal end 164 and a distal end 162. Proximal end 164 may be received within the larger diameter of first conduit portion 112 (for example, so that the joint diameter is not larger than the diameter of first conduit portion 112), while distal end 162 may surround the smaller diameter of second conduit portion 114. Bushing 160 may have a variable internal diameter, for example a larger diameter at distal end 162, such that second conduit portion 114 and bushing 160 provide a substantially uniform internal diameter. While not shown, bushing 160 may be also be arranged about the exterior of first conduit portion 112 and within second conduit portion 114. In non-limiting embodiments, bushing 160 may be bonded to first and second conduit portions 112, 114 by known methods described herein, and/or may be formed as a single piece with one or more of the conduit portions, for example by overmolding. In non-limiting embodiments, bushing 160 includes flange(s) 166, for example to provide stops for first fluid conduit 112. In non-limiting embodiments (not shown), bushing 160 is an additional conduit portion, that fills the gap when the inner diameter of first conduit portion 112 is larger than the outer diameter of second conduit portion 114.

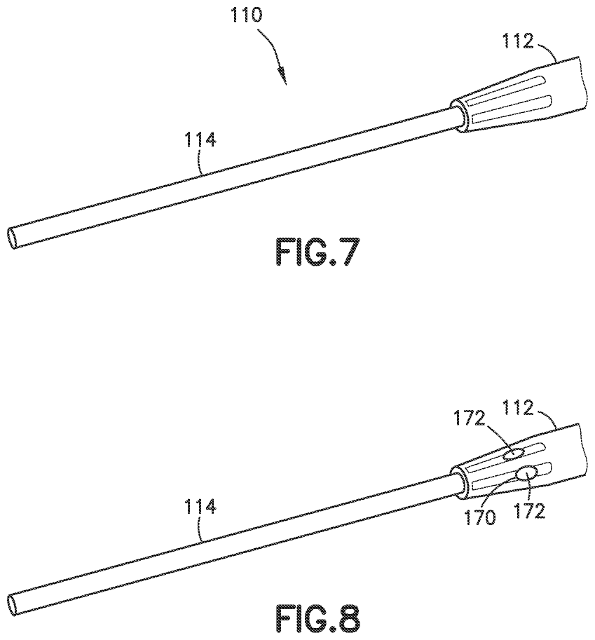

Turning to FIG. 7, shown is a non-limiting embodiment in which first conduit portion 112 and second conduit portion 114 are joined by overmolding. First fluid conduit 112 may have a tip region as described previously. Overmolding processes for joining two or more components are known to those of skill in the art.

Turning to FIG. 8, shown is a non-limiting embodiment in which first conduit portion 112 and second conduit portion 114 are joined through one or more interlock elements. For example, first conduit portion 112 may include one or more openings 170 at or near distal end thereof, and second conduit portion 114 may include one or more protrusions 172 at or near proximal end thereof, such that the one or more protrusions 172 may be received within the one or more openings 170, joining the conduit portions together. Opening(s) 170 and protrusion(s) 172 may be of any suitable number, size, and shape.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A blood draw device for withdrawing blood through an intravenous catheter assembly, the blood draw device comprising:
a housing;
a fluid conduit displaceably received within the housing and having a proximal end and a distal end, the fluid conduit comprising:
a first conduit portion at the proximal end of the fluid conduit, the first conduit portion having a proximal end, a distal end, and a first diameter; and
a second conduit portion at the distal end of the fluid conduit, the second conduit portion having a proximal end, a distal end, and a second diameter different from the first diameter,
wherein the fluid conduit is configured to be advanced from a first, proximal position in which the second conduit portion does not extend beyond a distal end of a catheter of the intravenous catheter assembly to a second, distal position in which the second conduit portion extends beyond a distal end of a catheter of the intravenous catheter assembly.

2. The blood draw device of claim 1, wherein the first diameter is larger than the second diameter.

3. The blood draw device of claim 1, wherein the distal end of the first conduit portion overlaps the proximal end of the second conduit portion.

4. The blood draw device of claim 3, wherein the first conduit portion includes, at the distal end thereof, a proximal to distal taper to a smaller diameter.

5. The blood draw device of claim 3, wherein the first conduit portion is joined to the second conduit portion by an adhesive, a weld, and/or by overmolding.

6. The blood draw device of claim 1, wherein the first conduit portion is joined to the second conduit portion with a stent.

7. The blood draw device of claim 6, wherein the stent is formed of nitinol.

8. The blood draw device of claim 6, wherein the stent is joined to the first conduit portion and/or the second conduit portion by an adhesive and/or a weld.

9. The blood draw device of claim 6, wherein the stent is arranged about an outer surface of the first conduit portion and the second conduit portion.

10. The blood draw device of claim 6, wherein the stent is received within the first conduit portion and the second conduit portion.

11. The blood draw device of claim 1, wherein the first conduit portion is joined to the second conduit portion with a double-sided wedge.

12. The blood draw device of claim 11, wherein the double-sided wedge comprises a first frustoconical end received within the first conduit portion and a second frustoconical end received within the second conduit portion.

13. The blood draw device of claim 11, wherein the double-sided wedge is joined to the first conduit portion and/or the second conduit portion by an adhesive and/or a weld.

14. The blood draw device of claim 1, wherein the first conduit portion is joined to the second conduit portion with a bushing having a proximal end and a distal end.

15. The blood draw device of claim 14, wherein the proximal end of the bushing is received within the first conduit portion, and the distal end of the bushing is arranged about an outer surface of the second conduit portion.

16. The blood draw device of claim 14, wherein the bushing further comprises one or more flanges, and wherein the distal end of the first conduit portion abuts the one or more flanges.

17. The blood draw device of claim 14, wherein at least a portion of an inner diameter of the bushing is the same as the second diameter.

18. The blood draw device of claim 14, wherein the bushing is joined to the first conduit portion and/or the second conduit portion by an adhesive and/or a weld.

19. The blood draw device of claim 1, wherein:
the first conduit portion comprises one or more openings in a sidewall thereof at or near the distal end thereof; and
the second conduit portion comprise one or more protrusions extending from a sidewall thereof at or near the proximal end thereof.

20. The blood draw device of claim 19, wherein the one or more protrusions are received within the one or more openings, thereby locking the first conduit portion to the second conduit portion.

21. The blood draw device of claim 1, wherein the second conduit portion comprises one or more openings in a sidewall thereof at or near the distal end thereof.

22. The blood draw device of claim 1, further comprising a third conduit portion arranged at the distal end of the second conduit portion, the third conduit portion comprising one or more openings in a sidewall thereof.

23. The blood draw device of claim 22, wherein the third conduit portion comprises a rounded distal end.

24. A catheter system comprising:
a catheter assembly comprising:
a catheter adapter, comprising a distal end, a proximal end, a lumen extending between the distal end and the proximal end; and
a catheter secured to the catheter adapter and extending distally from the catheter adapter, wherein the catheter comprises a distal end and a proximal end; and
a blood draw device for withdrawing blood through an intravenous catheter assembly, the blood draw device comprising:
a housing;
a fluid conduit displaceably received within the housing and having a proximal end and a distal end, the fluid conduit comprising:
a first conduit portion at the proximal end of the fluid conduit, the first conduit portion having a proximal end, a distal end, and a first diameter; and
a second conduit portion at the distal end of the fluid conduit, the second conduit portion having a proximal end, a distal end, and a second diameter different from the first diameter,
wherein the fluid conduit is configured to be advanced from a first, proximal position in which the second conduit portion does not extend beyond a distal end of a catheter of the intravenous catheter assembly to a second, distal position in which the second conduit portion extends beyond a distal end of a catheter of the intravenous catheter assembly.
